(19) United States
(12) Patent Application Publication
(10) Pub. No.: US 2024/0138377 A1
(43) Pub. Date: May 2, 2024

(54) IMPROVED ANIMAL COLLAR ASSEMBLY

(71) Applicant: CERES TAG PTY LTD, Red Hill (AU)

(72) Inventor: David Ian SMITH, Bunya (AU)

(21) Appl. No.: 18/547,397

(22) PCT Filed: Feb. 2, 2022
(86) PCT No.: PCT/AU2022/050126

§ 371 Date: Aug. 22, 2023

(30) Foreign Application Priority Data
Feb. 23, 2021 (AU) 2021900489

(51) Int. Cl. A01K 27/00; A01K 11/00; A01K 29/00

(52) U.S. Cl. CPC A01K 27/009; A01K 11/008; A01K 27/001; A01K 29/005

(57) ABSTRACT

An animal collar assembly for monitoring the animal's behaviour and/or animal's health, the collar comprising: an electronic module comprising a housing for enclosing electronic componentry therein, the electronic module comprising a first coupling arrangement for coupling to a collar strap adapted to be passed around a neck region of the animal; the collar strap comprising one or more radio frequency sensor arrays, wherein each sensor array further comprising: a radio frequency transmitter arranged for transmitting a signal towards a neck region of the animal; and a radio frequency receiver for receiving reflected signals from said neck region of the animal and generating a reflected signal dataset; a signal modification module operable to modify the reflected signal dataset; and a processor and control module housed in the electronic module, the processor and control module communicably coupled to the signal modification module, the processing and control system operable to generate a biometric measurement result for the animal based on the modified reflected signal dataset.

### TECHNICAL FIELD

The present invention relates to an improved animal collar assembly that is particularly, but not exclusively, well suited to pet animals such as dogs and cats.

### BACKGROUND

Any references to methods, apparatus or documents of the prior art are not to be taken as constituting any evidence or admission that they formed, or form part of the common general knowledge.

When animals, including pets such as dogs and cats, are unwell, early detection is difficult for pet owners. Whilst it is standard veterinary practice to check for the animal's vital signs such as heart rate, blood pressure and pulse, such checks are only carried out when the animal has become very sick and presented to the vet. Early detection is often not achieved yet is very important in order to achieve less suffering of the pet and less likelihood of a severe disease, which can develop if detection occurs late. Whilst many smart animal collars are currently known, most of these collars detect movements or require contacting probes to analyse animal health. Since most animals have a lot of fur, contact based electronic probes in animal collars have been generally unreliable. Therefore, there is a need to provide an improved animal collar that addresses some of the deficiencies of the prior art.

### SUMMARY OF INVENTION

In an aspect, the invention provides an animal collar assembly for monitoring an animal's behaviour and/or an animal's health, the collar including an electronic module including a housing for enclosing electronic componentry therein, the electronic module including a first coupling arrangement for coupling to a collar strap adapted to be passed around a neck region of an animal, and the collar strap including one or more radio frequency sensor arrays. Each sensor array further includes a radio frequency transmitter arranged for transmitting a signal towards a neck region of the animal and a radio frequency receiver for receiving reflected signals from said neck region of the animal and generating a reflected signal dataset. The electronic module also includes a signal modification module operable to modify the reflected signal dataset and a processor and control module housed in the electronic module, the processor and control module communicably coupled to the signal modification module, the processing and control system operable to generate a biometric measurement result for the animal based on the modified reflected signal dataset.

In an embodiment, the animal collar assembly further includes a delay module coupled to the radio frequency transmitter and operable to generate one or more delayed signals in accordance with one or more corresponding delay settings; wherein the radio frequency receiver is operable to receive one or more delayed reflected signals in accordance with said one or more corresponding delay settings and wherein the processor and control module are electrically coupled with the delay module to control said one or more delay settings.

In an embodiment, operation of the radio frequency transmitter and the receiver for each delay setting corresponds to an operating mode whereby reflected signals received from operation of the radio frequency arrays in a plurality of modes are processed by the processor to compute a biometric measurement result.

In an embodiment, the processor is programmed to process the biometric measurement result to generate one or more cardiovascular parameters for the animal.

In an embodiment, the animal collar assembly further includes an adjustment mechanism to control location of the sensor arrays for positioning the radio frequency sensor arrays to be located outside a reactive near-field with respect to the neck region of the animal.

In an embodiment, the sensor arrays are encapsulated within a flexible substrate of the collar strap to allow the sensory arrays to conform to contours of the animal's neck region.

In an embodiment, the processor is arranged to be in communication with a transmitter located in the housing of the electronic module to transmit the biometric measurement results over a wireless network.

In an embodiment, the animal collar assembly further includes a counterweight module having a weight that is equal to or greater than a weight of the electronic module, the counterweight module including a second coupling arrangement for coupling the counterweight module to the collar strap adapted to be passed around a neck region of the animal.

In an embodiment, the first coupling arrangement and/or the second coupling arrangement allows the counterweight module and the electronic modules to be coupled at spaced apart coupling locations along the length of the collar strap.

In an embodiment, the animal collar assembly further includes a rechargeable battery for powering electronic componentry in the electronic module and wherein each of the electronic modules further includes a connector for electrically connecting the rechargeable battery to a charging device.

In an embodiment, the electronic module is fixedly attached to the collar strap to prevent relative movement between the collar strap and the electronic module.

In an embodiment, the electronic module includes a location tracking device configured to wirelessly transmit animal location data from the electronic module to a remotely located device to provide remote animal location tracking capability for the remotely located device.

In an embodiment, the location tracking device is configured to be operationally inactive when the electronic module of the collar assembly is located within a pre-defined containment area.

In an embodiment, the processor that is operable in a normal containment operating mode to communicate location and animal health related data whilst the location tracking device is operationally inactive.

In an embodiment, the processor is in communication with a local short range transmission device to wirelessly transmit location and animal health related data to a receiving device located within the containment area.

In an embodiment, the processor is operable in a low power location tracking mode to process and transmit animal location related data when the electronic module of the collar assembly is located outside the pre-defined containment area.

In an embodiment, the location tracking device includes a satellite transmitter arranged to be in communication with the processor to transmit signals to one or more low earth orbit (LEO) satellites.

In an embodiment, the satellite transmitter transmits one-way data messages in relation to animal location from the electronic module to the one or more low earth orbit satellites.

In another embodiment, the satellite transmitter transmits two-way data messages in relation to animal location from the electronic module to the one or more low earth orbit satellites.

In an embodiment, the animal collar assembly further includes one or more additional sensor array with one or more sensor elements in communication with the processor, the additional sensor arrays including at least an accelerometer sensor, the accelerometer configured to measure at least one accelerometer-measured parameter of the animal from among: resting patterns, activity patterns, movement patterns and position patterns.

In an embodiment, the processor is configured to process the biometric measurement results by comparing the measured data with reference data to determine a likelihood of a specific health related condition in the animal and providing an indication of said likelihood on a display provided on the collar housing or on a remotely located device in communication with said electronic module.

In an embodiment, the determination of likelihood of the specific health related condition of the animal is carried out by comparing the measured parameters with one or more pre-set or pre-determined threshold values.

In an embodiment, the local or remotely located processors are configured to communicate with a user input interface for receiving user input to program measurement of one or more of said health related parameters.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is an in-use dorsal view of the animal collar assembly 100.

FIG. 2 is an in-use ventral view of the animal collar assembly 100.

FIG. 3 is a top perspective view of the animal collar assembly 100.

FIG. 4 is a top view of the animal collar assembly 100.

FIG. 5 is a schematic view of the strap portion of the collar assembly 100 that is adjacently located to the animal's neck.

FIG. 6 is a schematic view of the electronic module 120 showing various electronic components of the electronic module 120.

FIG. 7 is an isolated underside view of the electronic module 120.

FIG. 8 is an enlarged perspective view of the coupling member 140 and the electronic module 120 in an uncoupled configuration.

FIG. 9 is a schematic illustration of the collar assembly 100 shown during use in a containment zone Z.

FIG. 10 is a schematic illustration of the collar assembly 100 shown during use outside a containment zone Z.

### DETAILED DESCRIPTION OF PREFERRED EMBODIMENTS

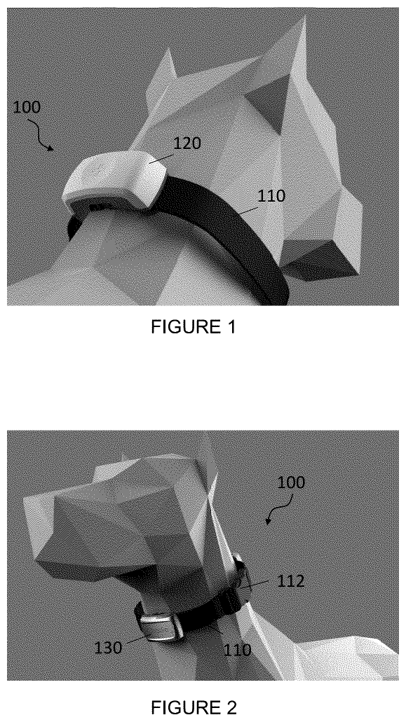

FIGS. 1 to 6 illustrate an animal collar assembly 100 in accordance with an embodiment of the present invention. The animal collar assembly 100 is suitable for monitoring an animal's behaviour and/or health and/or location. In the presently described embodiment, the animal collar assembly 100 is configured for performing a plurality of health monitoring functions which will be explained in the foregoing sections. The collar assembly 100 has been described with reference to being used as a dog collar. However, it would be understood that the collar assembly 100 may be used for similar monitoring and tracking other pet animals such as domestic cats without departing from the scope of the invention.

The collar assembly 100 includes a flexible strap 110 that can be passed around the animal's neck region and suitably tightened. The strap may be made from any suitable synthetic or natural material such as nylon or leather and formed as a continuous loop. Alternatively, a buckle such as buckle 112 may also be provided to fasten two ends of the strap 110 together to achieve a closed configuration. The collar assembly 100 includes an electronic module 120 which is coupled to the strap 110 via a coupling arrangement.

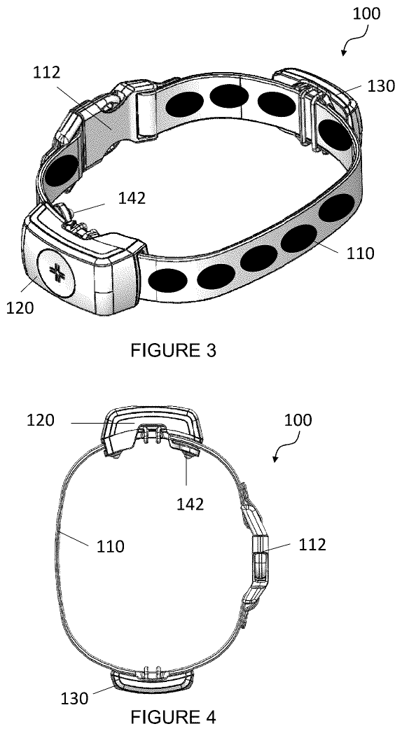

The flexible strap 110 includes a plurality of radio frequency sensor arrays 10. Each sensor array includes of a radio frequency (RF) transmitter 12 and a radio frequency (RF) receiver 14 best shown in FIGS. 3, 5 and 6. During use, when the collar strap is fastened around the animal's neck region, the RF transmitter 12 transmits a radio signal (preferably Super high frequency or Ultra high Frequency or Extremely High Frequency) towards the animal's arteries in the neck region. The RF receiver 14 is arranged to receive reflected signals from the animal's neck region to generate a reflected signal dataset. As will be evident from the foregoing sections, the configuration of the radio frequency sensor arrays 10 function to leverage an RF-based approach to non-invasively determine one or more biometric measurement results (e.g., cardiovascular parameters) thereby providing an indication of the animal's heath. The sensor arrays 10 in combination with a processor (housed in the electronic module 120) can also function to improve signal quality of one or more signals collected by the RF receivers 14, such as through processing collected signals Into a suitable form for generating accurate cardiovascular parameters based on the modified signals.

It is envisioned that the RF sensor transmitters 12 can dynamically improve the signal quality of signal datasets received by the RF receivers 14 for use in determining cardiovascular data. In order to continuously update parameters, a delay module 16 may be arranged in connection with the sensor arrays 10 for delaying signals, delay line settings, weighting parameters and control any other parameters affecting signal quality before, during, and/or after sessions of RF sensor 10 activity (e.g., transmission of incident signals towards an artery of the user, receipt of reflected signals, etc.). It should be understood that the sensor arrays 10 may be modified or configured to accommodate for variables affecting consistent signal quality, including user variations (e.g., different animal physiology, different motion, different ways of operating the RF system, etc.). RF system variations (e.g., different orientations of the RF sensor device, different arteries at which measurements are collected, etc.), and/or other variations.

The provision of a plurality of RF sensors 10 along the length of the collar strap 110 allows each sensor (or RF transmitter 12 and RF receiver 14 pair) to transmit and receive signals at different locations of an artery, and/or at different arteries. By way of a non-limiting example, a first RF sensor 10 may be used to collect reflected signals at a first location for an artery in the animal's neck and second RF sensor 10 may be used to collect reflected signals at a second location of the artery. Reflected signal datasets collected at different locations can then be used in evaluating body movement-related data (e.g., tissue movement-related data, respiration, heartbeat, arterial motion, stroke volume, pulse parameters such as pulse transit time and pulse wave velocity, etc.), from which cardiovascular parameters (e.g., heart beat metrics, blood pressure metrics, pulse rate metrics, physical activity metrics, metrics correlated with cardiovascular-related health, pulse oximetry metric, arterial metrics, respiration metrics, etc.) can be determined.

It is also envisioned that the RF sensors 10 may be capable of performing continuous monitoring of biometric parameters of the animal. For example, cardiovascular data such as heart rate, pulse and blood pressure data could be collected on a continuous basis with hundreds if not thousands of reflected signal datasets being generated every second. Alternatively, the RF sensors 10 could also be programmed to be dynamically triggered (e.g., in response to detecting an inactive user state based on motion data collected by other additional sensors 20 (such accelerometer type sensors as will be discussed in the foregoing sections).

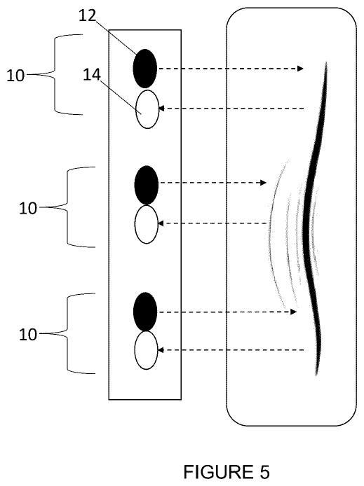

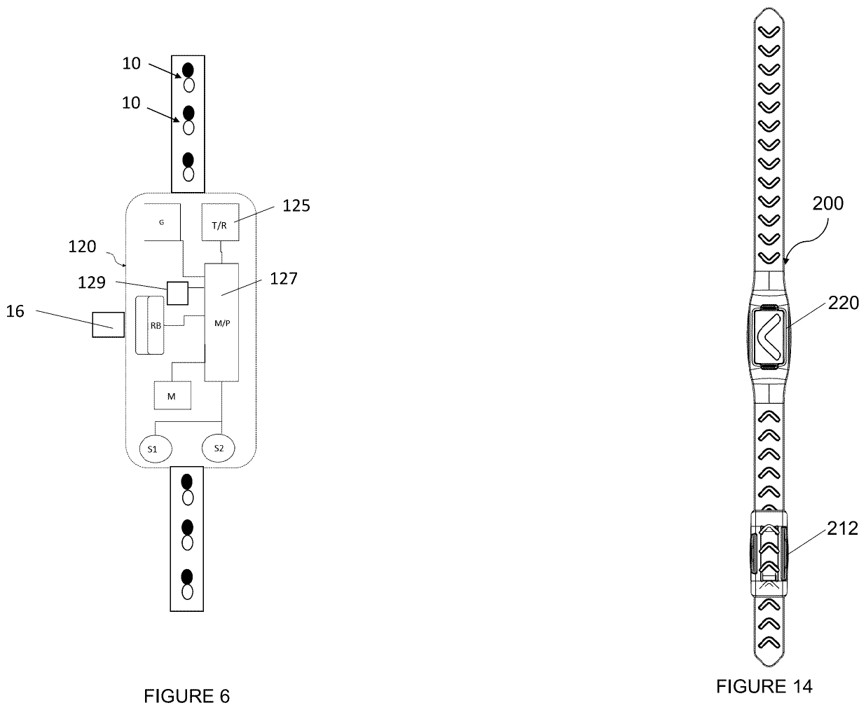

Referring to FIGS. 5 and 6 in particular, each RF sensor 10 located along the collar strap 110 configured to transmit RF signals (e.g., incident pulse signals) towards the neck region of the animal, and to receive reflected signals for generating a reflected signal dataset. A signal modification module 16 may be used to modify the reflected signal dataset. The electronic module 120 houses a processor 121; a controller 123 that is arranged to be in communication with the RF sensors 10 and the signal modification module 16. The processor 121 and the controller 123 are arranged to generate cardiovascular parameters for the animal based on the modified reflected signal dataset received by the RF receiver 14 of each RF sensor 10. It would also be understood that each RF sensor 10 is arranged to communicate with a signal generator 18.

The RF sensor 10 may be preferably constructed with flexible materials to conform to the contour of the animal's neck region. It is also envisioned that these RF sensors 10, namely the electronic componentry may be encapsulated in a flexible material to allow the RF sensors 10 to better conform to the animal's contours. In alternative embodiments, rigid materials may also be used for constructing the RF sensor array 10.

Furthermore, one or more adjustment mechanisms may be used to control to control location of the sensor arrays 10 for positioning the radio frequency sensor arrays 10 to be located outside a reactive near-field with respect to the neck region of the animal. By way of example, a mechanism may be provided to fixing the distance between the RF sensors 10 and a target region of the animal's neck. In this regard, the buckle 112 may be utilised to ensure that the target neck region of the animal always lies outside the near-field of the RF transmitter 12 and RF receiver 14 pair.

Each of the RF sensors 10 is in communication with the processor 121 that is housed in the electronic module's housing 120. The processor 121 is also arranged to be in signal communication with a data transmitter 125 to transmit data or signals associated with processed reflected signals received by the RF sensors 10 in the collar strap 110. As shown best in FIG. 1, during use, it is considered desirable if not ideal to maintain the location of the electronic module 120 behind (or above) the ears of the animal such that the electronic module 120 is substantially equidistant from each ear of the animal. The mounting location of the electronic module 120 is substantially maintained by providing the counterweight module 130 that is also coupled with the strap 110 at a spaced apart location from the electronic module 120 in a manner such that during use, the counterweight module 130 is located along a ventral region of the animal's body. Advantageously, the inventor has found that providing the counterweight module 130 that has a weight that is at least substantially equal to or preferably greater than the weight of the electronic module 120 causes the heavier counterweight module 130 to be maintained at a ventral region of the animal's body as a result of which the relatively lighter electronic module 120 remains behind the animal's ears along a dorsal portion of the animal's body even during prolonged periods of use. Maintaining the position of the electronic module 120 improves connectivity between data transmitters in the electronic module 120 and any satellites as will be explained in further detail. Therefore, providing the counterweight module 130 prevents the electronic module 120 from slipping towards a ventral region of the animal's body. The counterweight module 130 may be utilised for performing other secondary functions. In one useful embodiment, the counterweight module 130 may be provided with an outwardly visible surface to provide useful indicia such as (but not limited to) the animal's name to assist in correctly identifying the animal. Other useful information in relation to the pet animal's owner may also be provided on the outer surface of the counterweight module 130.

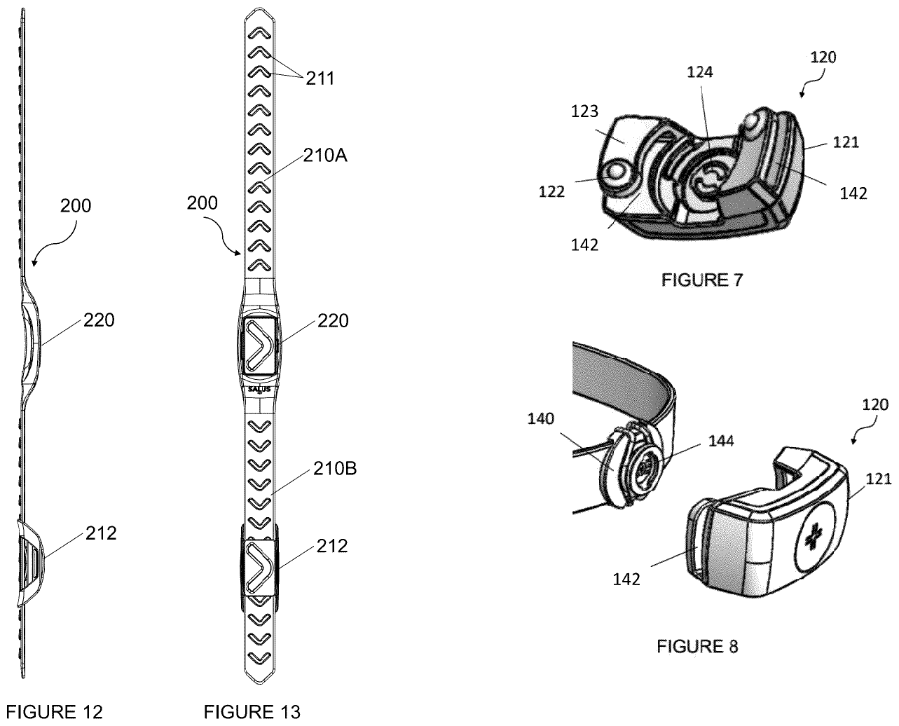

The coupling arrangement (best shown in FIGS. 7 and 8) for the electronic module 120 also includes a coupling member 140 that is affixed to the collar strap 110. The coupling member 140 includes a receiving portion 142 for receiving the collar strap 110 and a locking formation 144 for receiving and coupling with the electronic module housing 120. In the preferred embodiment, the module housing 120 includes a recessed locking portion 124 (located in between the tabs 125) with locking channels that cooperate with the locking formation 144 of the coupling member 140 such that twisting the module housing 120 in a clockwise or anti-clockwise direction results in the locking channels receiving the locking formation 144 of the coupling member 140. It would be understood that this manner of coupling the module housing 120 with the coupling member 140 is not limiting. For example, in alternative embodiments, the locking formations 144 might be provided within the recessed locking portion 124 and locking channels may be provided in the coupling member 140 without departing from the scope of the invention as described.

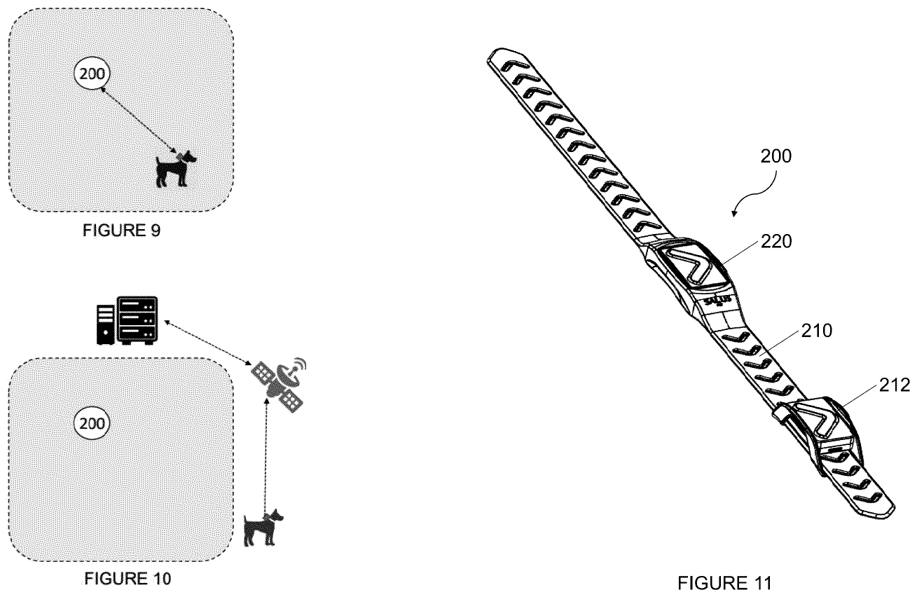

Turning to FIG. 6, the electronic module 120 includes several electrical components including a microprocessor (M/P) which is in communication with an on-board non-volatile memory device (M). The microprocessor (M/P) is adapted for short-range wireless communication with a docking station 200 via a transceiver (LTR) that is arranged to be operatively coupled with the microprocessor (M/P). As shown particularly well in FIGS. 9 and 10, the docking station 200 is continuously in communication with the electronic module 120 (mounted on the animal) when the animal is within a predefined containment zone (Z). In short, the transceiver (LTR) periodically communicates with the docking station 200 to determine and confirm if the animal is within the pre-defined containment zone (Z). Such a mode of operation may be deemed to be a first operating configuration and may be referred to as a “containment mode” throughout the specification. The electronic module 120 may also house a battery pack including rechargeable batteries RB which may be recharged on a periodic basis.

During operation in the containment mode, the microprocessor (M/P) may receive sensory data from an array of additional sensors such as (but not limited to) S1 and S2 in communication with the microprocessor (M/P). The additional sensor array may include an accelerometer configured to measure at least one accelerometer-measured bioparameter of the animal from among: resting patterns, activity patterns, movement patterns, position patterns, noise and sound patterns, lameness and scratching, and the non-accelerometer sensor configured to measure at least one of the following non-accelerometer-measured bioparameters of the pet animal: temperature, pulse rate, respiration rate. The at least two sensor elements may includes at least three or at least four or at least five or at least six or at least seven (or more) sensor elements distributed at different locations within the electronic module housing 120. The on board microprocessor (M/P) may receive data values from the one or more sensors (S1, S2) of the additional sensor array and the RF sensor array 10 and compare these values with reference data to determine a likelihood of a specific health related condition in the animal and providing an indication of said likelihood on a display provided on the collar housing or on a remotely located device such as the docking station 200 or a remotely located device (such as a mobile computing device including a smartphone or tablet or any other computing device) in communication with said electronic module 120. In this regard, the remotely located device may communicate directly with the docking station 200 via the internet or any other communication network. In some instances, the on-board memory device (M) may include some reference values for comparison with data received from the sensors S1 and S2. In other scenarios, the sensor data may be transmitted to the docking station 200 via the transceiver (LTR) and comparative processing may be carried at a remote processing location by utilising a server or computers communicating with the docking station 200.

The docking station 200 may be used for updating the firmware installed on the microprocessor (M/P). The updates may be carried out by either transferring one or more update files from the docking station 200 whilst the electronic module 120 is docked on the docking station 200. Alternatively, or additionally, the updates from the docking station 200 may be transmitted to the electronic module 120 wirelessly.

FIGS. 11 to 14 illustrate yet another embodiment of a collar assembly 200 in accordance with the present invention. Like reference numerals denote like features which have been described in previous sections. The collar assembly 200 is also suitable for monitoring the animal's behaviour and/or health and/or location. In the presently described embodiment, the animal collar assembly 200 is configured as a dog collar. However, the animal collar assembly 200 is not limited to such use.

The collar assembly 200 also includes a flexible strap 210 that can be passed around the animal's neck region and suitably tightened. The strap 210 may be made from any suitable synthetic or natural material and formed to be fastened and provide a continuous loop. A buckle 212 is provided to fasten two ends of the strap 210 together to achieve a closed configuration. The collar assembly 200 also includes an electronic module 220 which is fixedly coupled to the strap 210 at two ends of the housing 220 via a fixed coupling arrangement. The upper surface of the strap 210 includes a plurality of engagement portions 211 that are positioned in a spaced apart arrangement along the length of the strap segments 210A and 210B that may be engaged by the buckle 212 to form the closed loop. The plurality of engagement portions 211 may be formed integrally within the flexible strap 210 or alternatively may be embedded therewithin.

The flexible strap 210 also includes a plurality of the radio frequency sensor arrays 10 which have been described in the previous sections. Each sensor array 10 includes of a radio frequency (RF) transmitter 12 and a radio frequency (RF) receiver 14 best shown in the conceptual FIGS. 5 and 6. During use, when the collar strap 210 is fastened around the animal's neck region, the RF transmitter 12 transmits a radio signal (preferably Super high frequency or Ultra high Frequency or Extremely High Frequency) towards the animal's arteries in the neck region. The RF receiver 14 is arranged to receive reflected signals from the animal's neck region to generate a reflected signal dataset. As will be evident from the foregoing sections, the configuration of the radio frequency sensor arrays 10 function to leverage an RF-based approach to non-invasively determining one or more biometric measurement results (e.g., cardiovascular parameters) thereby providing an indication of the animal's heath. The sensor arrays 10 work in combination with a processor housed in the electronic module 220. The processor within the electronic module 220 works in substantially the same manner as previously described.

Each RF sensor 10 located along the collar strap 210 transmits RF signals (e.g., incident pulse signals) towards the neck region of the animal and receives reflected signals for generating a reflected signal dataset. A signal modification module 16 may modify the reflected signal dataset. The electronic module 220 houses the processor 121 and the controller 123 that is arranged to be in communication with the RF sensors 10 and the signal modification module 16. The processor 121 and the controller 123 are arranged to generate biometric parameters such as but not limited to cardiovascular parameters for the animal based on the modified reflected signal dataset received by the RF receiver 14 for each RF sensor 10.

The RF sensors 10 are embedded within the thickness of the flexible strap 210 and preferably encapsulated in flexible material to better conform to the animal's contours. The plurality of RF sensors 10 in the strap segments 210A and 201B may be located at a desirable location adjacent a target physiological region of the animal's neck. The buckle 212 in combination with the engagement portions 211 allows for more precise positioning of the RF sensors 10 around the animal's neck region. Each of the RF sensors 10 is in communication with the processor 121 that is housed in the electronic module's housing 220. The processor 121 is also arranged to be in signal communication with the data transmitter 125 to transmit data or signals associated with processed reflected signals received by the RF sensor arrays 10 in the flexible strap 210. In the preferred embodiment, the RF sensors 10 in the strap segments 210A and 2104 are connected to the various components of the electronic module 220 by flexible and flattened conductors embedded within the collar strap segments 210A and 210B.

Once again, it is considered desirable if not ideal to maintain the location of the electronic module 220 behind (or above) the ears of the animal such that the electronic module 220 is substantially equidistant from each ear of the animal. The mounting location of the electronic module 220 is substantially maintained by providing a counterweight module which forms a part of the buckle 212. the counterweight buckle 212 is also coupled with the flexible strap 210 at a spaced apart location from the electronic module 220 in a manner such that during use, the counterweight buckle 212 is located along a ventral region of the animal's body. The counterweight buckle 212 either alone or in combination with additional counterweight bodies (not shown) forms a counterweight module 230 so that the counterweight module 230 has a weight that is at least substantially equal to or preferably greater than the weight of the electronic module 220 which causes the heavier counterweight buckle 212 to be maintained at a ventral region of the animal's body as a result of which the relatively lighter electronic module 220 remains behind the animal's ears along a dorsal portion of the animals' body even during prolonged periods of use. Maintaining the position of the electronic module 220 improves connectivity between data transmitters in the electronic module 220 and any satellites as will be explained in further detail. Therefore, providing the counterweight buckle 212 prevents the electronic module 220 from slipping towards a ventral region of the animal's body. The counterweight buckle module 230 may also be utilised for performing other secondary functions. In one useful embodiment, the counterweight module 230 may be provided with an outwardly visible surface to provide useful indicia such as (but not limited to) the animal's name to assist in correctly identifying the animal. Other useful information in relation to the pet animal's owner may also be provided on the outer surface of the counterweight module 230.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. An animal collar assembly for monitoring the animal's behaviour and/or animal's health, the collar comprising:
an electronic module comprising a housing for enclosing electronic componentry therein, the electronic module comprising a first coupling arrangement for coupling to a collar strap adapted to be passed around a neck region of the animal;
the collar strap comprising one or more radio frequency sensor arrays, wherein each sensor array further comprising:
a radio frequency transmitter arranged for transmitting a signal towards one or more arteries in a neck region of the animal; and
a radio frequency receiver for receiving reflected signals from said neck region of the animal and generating a reflected signal dataset;
a signal modification module operable to modify the reflected signal dataset to generate a modified reflected signal dataset; and
a processor and control module housed in the electronic module, the processor and control module communicably coupled to the signal modification module, the processing and control system operable to generate a biometric measurement result for the animal based on the modified reflected signal dataset.

2. An animal collar assembly in accordance with claim 1 wherein the signal modification module is positioned with the housing of the electronic module.

3. An animal collar assembly in accordance with claim 1 comprising a plurality of said radio frequency sensor arrays located along the length of the collar strap.

4. An animal collar assembly in accordance with claim 1 further comprising a delay module coupled to the radio frequency transmitters and operable to generate one or more delayed signals in accordance with one or more corresponding delay settings; wherein the radio frequency receivers are operable to receive one or more delayed reflected signals in accordance with said one or more corresponding delay settings and wherein the processor and control module are electrically coupled with the delay module to control said one or more delay settings.

5. An animal collar assembly in accordance with claim 1 wherein operation of the radio frequency transmitters and the receivers for each delay setting corresponds to an operating mode whereby reflected signals received from operation of the radio frequency arrays in a plurality of modes are processed by the processor to compute a biometric measurement result.

6. An animal collar assembly in accordance with claim 1 wherein the processor is programmed to process the biometric measurement result to generate one or more cardiovascular parameters for the animal.

7. An animal collar assembly in accordance claim 1 further comprising an adjustment mechanism to control location of the sensor arrays for positioning the radio frequency sensor arrays to be located outside a reactive near field with respect to the neck region of the animal.

8. An animal collar assembly in accordance with claim 1 wherein the processor is arranged to be in communication with a transmitter located in the housing of the electronic module to transmit the biometric measurement results over a wireless network.

9. An animal collar assembly in accordance with claim 1 further comprising a counterweight module having a weight that equal to or greater than weight of the electronic module, the counterweight module comprising a second coupling arrangement for coupling the counterweight module to the collar strap adapted to be passed around a neck region of the animal.

10. An animal collar assembly in accordance with further comprising a counterweight module having a weight that equal to or greater than weight of the electronic module, the counterweight module comprising a second coupling arrangement for coupling the counterweight module to the collar strap adapted to be passed around a neck region of the animal.

11. An animal collar assembly in accordance with claim 10 wherein the second coupling arrangement allows the counterweight module and the electronic modules to be coupled at a plurality of spaced apart coupling locations along the length of the collar strap.

12. An animal collar assembly in accordance with claim 1 a rechargeable battery for powering electronic componentry in the electronic module and wherein the electronic housing further comprises a connector for electrically connecting the rechargeable battery to a charging device.

13. An animal collar assembly in accordance with claim 1 wherein the electronic module is fixedly attached to the collar strap to prevent relative movement between the collar strap and the electronic module.

14. An animal collar assembly in accordance with claim 1 wherein the electronic module comprises a location tracking device configured to wirelessly transmit animal location data from the electronic module to a remotely located device to provide remote animal location tracking capability for the remotely located device.

15. An animal collar assembly in accordance with claim 14 wherein the location tracking device is configured to be operationally inactive when the electronic module of the collar assembly is located within a pre-defined containment area.

16. An animal collar assembly in accordance with claim 15 wherein the processor is operable in a normal containment operating mode to allow the transmitter to transmit the biometric measurement result for the animal to the receiving device via short range communication whilst the location tracking device is operationally inactive.

17. An animal collar assembly in accordance with claim 15 wherein the processor is operable in a low power location tracking mode to process and transmit animal location related data when the electronic module of the collar assembly is located outside the pre-defined containment area and simultaneously stop or delay operation of the sensor arrays.

18. An animal collar assembly in accordance with any one of claims 14 to 17 claim 14 wherein the location tracking device comprises a satellite transmitter arranged to be in communication with the processor to transmit signals to one or more low earth orbit (LEO) satellites via direct satellite communication.

19. An animal collar assembly in accordance with claim 18 wherein the satellite transmitter transmits one-way data messages in relation to animal location from the electronic module to the one or more low earth orbit satellites.

20. An animal collar assembly in accordance with claim 18 wherein the satellite transmitter transmits two-way data messages in relation to animal location from the electronic module to the one or more low earth orbit satellites.

21. (canceled)

22. (canceled)

23. (canceled)

24. (canceled)
